(19) United States
(12) Patent Application Publication
(10) Pub. No.: US 2002/0193762 A1
(43) Pub. Date: Dec. 19, 2002

(54) Disposable urinal system

(76) Inventor: Kristen V. Suydam, Phoenix, AZ (US)

Correspondence Address: STONEMAN LAW OFFICES, LTD, 3113 NORTH 3RD STREET, PHOENIX, AZ, 85012, US

(21) Appl. No.: 09/883,678

(22) Filed: Jun. 18, 2001

(51) Int. Cl. A61M 1/00; A61F 5/44; A61B 5/00; B65D 81/00

(52) U.S. Cl. 604/327; 604/355; 600/574

(57) ABSTRACT

A hand-held disposable portable urinal device particularly well-suited for female use. The easy-to-use device has a leak and splash resistant soft upper seal attached to a retractable funnel, which easily folds back into a sealable bag. The funnel has a neck portion which allows the user to comfortably hold the urinal close to the ureter during use. The disposable urinal bag is odor- and vapor-sealable, and has a hydrophobic polymer containing inner bag for absorbing the urine. In addition, the disposable urinal has a deodorizer activated by the urine upon contact. The disposable urinal is easily transported in a folded state, and preferably comes three in a box. After use, the disposable urinal may be sealed and thrown away.

### BACKGROUND

This invention relates to providing a system for an improved portable urinal device which is both sealable and disposable. The elimination device is particularly well-suited for female use, although it may be used by males as well.

Typically, people sometimes experience the need to eliminate urine when access to a toilet or other such restroom facility is not readily available. This may occur during traveling, such as in an automobile or airplane, camping, hiking or a variety of other such activities. Further, persons having physical disabilities or health concerns, such as surgical procedures, may not be able to access a restroom or such permanent urinal. Public restrooms are often unsanitary, and persons may desire to use a more sanitary elimination device.

Existing such devices, and including makeshift cans, bottles, etc., may be cumbersome and difficult to use. For a female, it may be difficult to grasp such a device and hold it near the body without leakage or unwanted splashing of the urine during elimination; and disposal may be difficult.

There is a continuing need for a sanitary, disposable urinal system that is comfortable, easy to use, doesn't leak, doesn't smell foul, and is easily stored both prior to use and prior to disposal after use.

### OBJECTS OF THE INVENTION

A primary object and feature of the present invention is to provide a device which overcomes the above-mentioned problems with prior devices.

It is a further object and feature of the present invention to provide a portable urinal system which is both sealable and disposable.

It is a further object and feature of the present invention to provide such a system which is easily stored both prior to use and prior to disposal after use.

It is an even further object and feature of the present invention to provide such a system which may be hand-held against the body and is comfortable, easy to use and doesn't leak.

It is yet another object and feature of the present invention to provide such a system which has self-contained wiping tissue for clean-up after elimination.

A further primary object and feature of the present invention is to provide such a system which is efficient, inexpensive, and handy. Other objects and features of this invention will become apparent with reference to the following descriptions.

### SUMMARY OF THE INVENTION

In accordance with a preferred embodiment, this invention provides a disposable, hand-holdable, portable urinal system for holding the urine of a user during and after urination, comprising: a first bag comprising: substantially a liquid- and odor-impenetrable material, at least one hollow interior, and at least one opening comprising a closure which is structured and arranged to seal and un-seal such at least one opening, wherein such first bag is structured and arranged to hold the urine; and a funnel, attached to an interior of such first bag, comprising a liquid- and odor-impenetrable material, and an end portion having a rim portion, wherein such funnel is structured and arranged to wholly fit into such first bag, be pullable through such opening from such first bag into a position to direct flow of the urine from such funnel into such first bag, and be returnable wholly through such opening into such first bag after urination, wherein such end portion is structured and arranged to permit a single hand of the user to hold such funnel so as to position such rim portion without substantial interference to a flow of the urine through such funnel, and wherein such rim portion is structured and arranged to closely fit the genitals of a female user and comprises a soft absorbent seal along a periphery of such rim portion structured and arranged to substantially prevent leaking of the urine, during urination, from such periphery of such rim portion.

Further, it provides such a system further comprising a urine-absorber located within such first bag. And, it provides such a system wherein such urine-absorber is a gellable hydrophilic material. It also provides such a system further comprising: a second bag located within such first bag; wherein such urine-absorber is contained within such second bag; and wherein such second bag is fluid permeable. Even further, it provides such a system further comprising a deodorizer contained within such second bag and structured and arranged to be activated by the urine. And, it provides such a system further comprising a deodorizer contained within such first bag and structured and arranged to be activated by the urine.

Even further, it provides such a system further comprising: a holder attached to an exterior of such first bag, wherein such holder is structured and arranged to hold at least one wiping element. It also provides such a system further comprising: a holder attached to an exterior of such first bag, wherein such holder comprises at least one wiping element. And, it provides such a system wherein such at least one wiping element comprises at least one tissue.

Moreover, it provides such a system wherein such at least one wiping element comprises at least one moistened towelette. And, it provides such a system wherein such closure comprises a flexible closure. And, it provides such a system wherein such funnel is foldable. Still further, it provides such a system wherein such rim portion comprises a substantially cotton-fiber material. It also provides such a system wherein such rim portion comprises a stiffener structured and arranged to substantially maintain shape during urination use.

Additionally, it provides such a system comprising: a holder attached to an exterior of such first bag; wherein such holder comprises at least one wiping element selected from the group consisting of a moist towlette a wiping tissue; and wherein such rim portion comprises a substantially cotton-fiber material; and wherein such rim portion comprises a stiffener structured and arranged to substantially maintain shape during urination use.

In accordance with another preferred embodiment, this invention provides a disposable, hand-holdable, portable urinal system for holding the urine of a user, during and after urination, comprising: a sealable holding means for holding the urine; funnel means, attached to an interior of such holding means, for funneling the urine into the holding means; wherein such funnel means comprises rim means for cushioned leak-sealing of such funnel means adjacent genitals of the user during urination; and removal means for implementing extension of such funnel means from such holding means and stowage of such funnel means within such holding means.

Furthermore, it provided such a system further comprising: kit means for packaging for sale in a single package at least one such holding means, at least one such funnel means, and at least one such removal means. It also provides such a system comprising a plurality of products, within one such kit means, each comprising one such holding means, one such funnel means, and one such removal means.

In accordance with yet another preferred embodiment, this invention provides a disposable, hand-holdable, portable urinal system for holding the urine of a user, during and after urination, comprising: a sealable holder structured and arranged to hold the urine; a funnel, attached to an interior of such holder, structured and arranged to funnel the urine into such holder; wherein such funnel comprises a rim portion structured and arranged to provide cushioned leak-sealing of such funnel adjacent genitals of the user during urination; and a removal system structured and arranged to implement extension of such funnel from such holder for use during urination and stowage of such funnel within such holder before and after.

### BRIEF DESCRIPTION OF THE DRAWINGS

FIG. 1 is a perspective view, partially in section, of the disposable urinal system in use by a female according to a preferred embodiment of the present invention.

FIG. 2 is a perspective view of the disposable urinal system with the funnel portion in the ready-to-use position according to a preferred embodiment of the present invention.

FIG. 3 is a perspective view of the disposable urinal system in the sealed and stored position according to a preferred embodiment of the present invention.

FIG. 4 is a perspective cut-away view, partially in section, of the disposable urinal system illustrated with the funnel portion in the ready-to-use position according to a preferred embodiment of the present invention.

FIG. 5 is a sectional view through the section 5-5 of FIG. 4.

FIG. 6 is a perspective cut-away view, partially in section, of the disposable urinal system in the sealed and stored position according to a preferred embodiment of the present invention.

FIG. 7 is a sectional view through the section 7-7 of FIG. 2.

FIG. 8 is a perspective view of the disposable urinal system illustrating three disposable urinals stored in a packaging container.

FIG. 9 is a perspective view illustrating the disposable urinal system being disposed.

### DETAILED DESCRIPTION OF A PREFERRED EMBODIMENT OF THE INVENTION

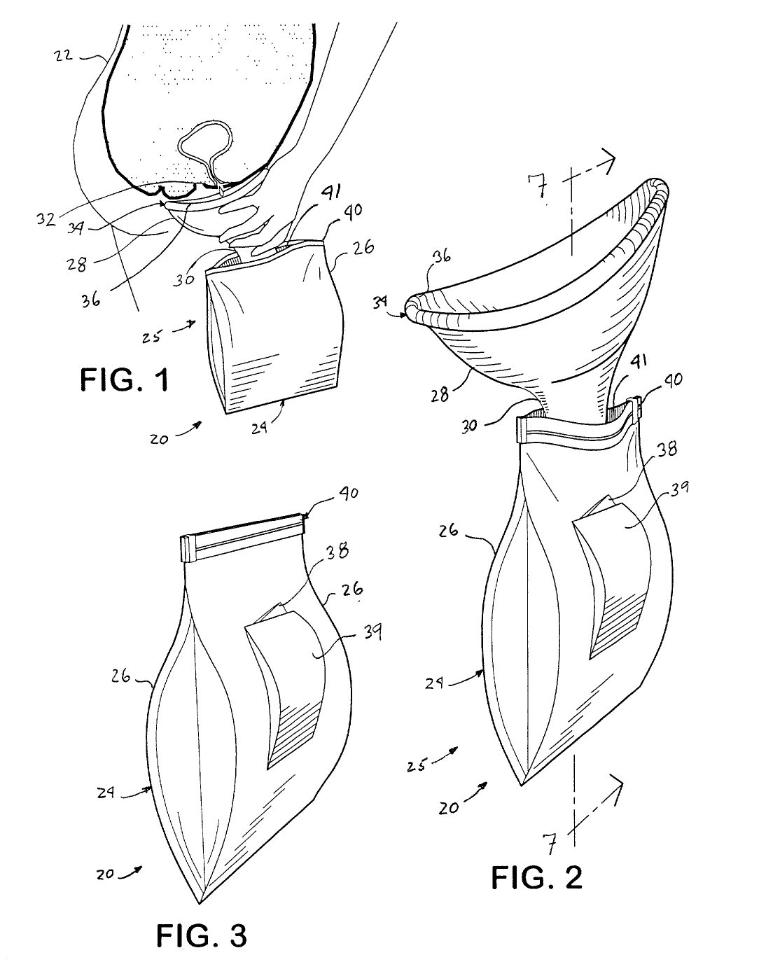

FIG. 1 is a perspective view, partially in section, of the disposable urinal system 20 in use by a female user 22 according to a preferred embodiment of the present invention. Preferably, a disposable urinal 24 comprises a first sealable outer bag 26 (embodying herein a first bag; and embodying herein a sealable holder structured and arranged to hold the urine; and embodying herein a sealable holding means for holding the urine) and an integral, peferably oval-shaped rim, funnel 28 (embodying herein a funnel, attached to an interior of such first bag; and embodying herein a funnel, attached to an interior of such holder, structured and arranged to funnel the urine into such holder; and also embodying herein funnel means, attached to an interior of such holding means, for funneling the urine into the holding means). Preferably, the funnel 28 is shaped such that the user 22 may easily grasp the funnel 28 and neck portion 30 without interfering with the flow of the urine through the funnel 28 (embodying herein an end portion; wherein such end portion is structured and arranged to permit a single hand of the user to hold such funnel so as to position such rim portion without substantial interference to a flow of the urine through such funnel). In use by females, the funnel 28 is preferably held as shown, and placed between the legs and adjacent the body of the user 22, preferably with the urethra 32 of the user 22 directly above the funnel 28. Preferably, funnel 28 comprises a rim 34 (embodying herein a rim portion). Preferably, rim 34 comprises a soft absorbent seal 36 along the outer periphery of the funnel rim 34 that functionally prevents leaking (as well as adding to comfort) during urine voiding when held closely to the body, between the legs of the user 22, as shown (the above described arrangement embodying herein that such rim portion is structured and arranged to closely fit the genitals of a female user and comprises a soft absorbent seal along a periphery of such rim portion structured and arranged to substantially prevent leaking of the urine, during urination, from such periphery of such rim portion; and embodying herein wherein such funnel comprises a rim portion structured and arranged to provide cushioned leak-sealing of such funnel adjacent genitals of the user during urination; and also embodying herein wherein such funnel means comprises rim means for cushioned leak-sealing of such funnel means adjacent genitals of the user during urination).

FIG. 2 is a perspective view of the disposable urinal system 20 with the funnel 28 of the disposable urinal 24 in the ready-to-use position 25 extending upward and outward from the bag 26, as shown, according to a preferred embodiment of the present invention. Preferably, the disposable urinal 24 comprises a tissue holder 39 which is preferably attached to the exterior of bag 26 as shown (embodying herein a holder attached to an exterior of such first bag, wherein such holder is structured and arranged to hold at least one wiping element). Preferably, the tissue holder 39 is formed as an integral part of bag 26. Preferably, tissue holder 39 further comprises tissue 38 which is preferably used by user 22 to clean-up after voiding. Tissue 38 is preferably a moist towlette of the type well-known by those knowledgeable in the art; however, under appropriate circumstances, other types of tissue 38 or wipes may suffice (this arrangement embodying herein a holder attached to an exterior of such first bag; wherein such holder comprises at least one wiping element selected from the group consisting of a moist towlette, a wiping tissue; and embodying herein wherein such at least one wiping element comprises at least one tissue; and also embodying herein wherein such at least one wiping element comprises at least one moistened towelette).

FIG. 3 is a perspective view of the disposable urinal system 20 illustrating the disposable urinal 24 in the sealed and stored position 27 according to a preferred embodiment of the present invention. Preferably, the disposable urinal 24 is stored prior to use, and also after use, in the illustrated manner. Preferably, bag 26 comprises a sliding closure device 40 arranged to seal and un-seal opening 41 (this arrangement embodying herein at least one opening comprising a closure which is structured and arranged to seal and un-seal such at least one opening). Preferably, sliding closure device 40 is a flexible closure (embodying herein wherein such closure comprises a flexible closure) of the type well known in the art for flexible plastic containers which provides a strong closure with an effective vapor and moisture barrier (e.g. a Ziplock®-type closure). Under appropriate circumstances, other suitable arrangements may suffice. FIG. 6 further illustrates the bag 26 contents in the stored position 27 (also a disposable condition 29 when sealed).

FIG. 6 is a perspective cut-away view, partially in section, of the disposable urinal system 20 in the sealed and stored position 27 according to a preferred embodiment of the present invention. Preferably, funnel 28 folds into bag 26 as shown (embodying herein wherein such funnel is foldable), such that bag 26 may be easily sealed with sliding closure device 40. Preferably, funnel 28 folds into bag 26 by folding the funnel longitudinally and then pushing the funnel 28 directly and centered, into bag 26 (see FIG. 7 for side view).

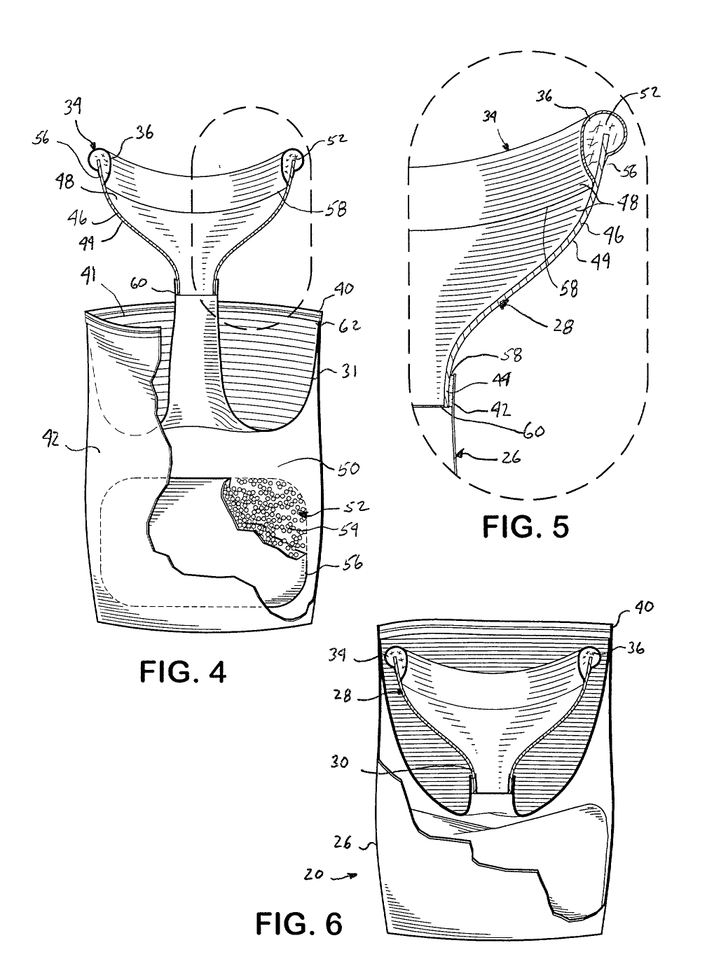

FIG. 4 is a perspective cut-away view, partially in section, of the disposable urinal system 20 with the funnel 28 of the disposable urinal 24 in the ready-to-use position 25 extending upward and outward from the bag 26, as shown, according to a preferred embodiment of the present invention. Preferably, bag 26 comprises an outer layer 42, which is a liquid- and odor-impenetrable material, such as plastic (embodying herein a first bag comprising: substantially a liquid- and odor-impenetrable material). Preferably, the outer layer 42 is opaque in color; however, any color that would block the view of the contents would be sufficient (it is preferable that the bag contents not be visible). Preferably, funnel 28 comprises an outer layer 44 which is also liquid- and odor-impenetrable material, such as plastic (embodying herein a funnel, attached to an interior of such first bag, comprising a liquid- and odor-impenetrable material). Preferably, funnel 28 also comprises an upper, inside layer 46, which is a liquid-impenetrable material, such as plastic. Preferably, in one embodiment, upper inside layer 46 also comprises an exterior cellulose fiber layer 48 to assist in avoiding any splashing of the urine as it is voided into the disposable urinal 24 device by the user 22. Preferably, cellulose fiber layer 48 extends from the rim 34 downward to the neck portion 30. Under appropriate circumstances, other dimensions of the cellulose fiber layer 48 may suffice. In another embodiment, the outer layer 44 and the upper inside layer 46 are a single layer of liquid impenetrable material, such as plastic. In this embodiment, the rim 34 is a separate attachment comprising a soft absorbent seal 36 and a cellulose fiber layer 48, as shown. Preferably, the cellulose fiber layer 48 acts as a stiffener and assists in maintaining the preferred oval shape of the rim 34, especially during urination (this arrangement embodying herein wherein such rim portion comprises a stiffener structured and arranged to substantially maintain shape during urination use). In addition, funnel 28 is connected to bag 26 by a flexible extended portion 31. Preferably, flexible extended portion 31 is soft flexible plastic material sheeting. Under appropriate circumstances, other materials may suffice.

Preferably, the voided urine travels in a downward direction along the funnel 28 by gravity. Preferably, the urine is emptied into the main body 50 of the bag 26. Preferably, bag 26 further comprises a hydrophilic material 52 (embodying herein a urine-absorber located within such first bag; and wherein such urine-absorber is a gellable hydrophilic material). Preferably, hydrophilic material 52 is a hydrophilic polymer of the type that absorbs fluid entering bag 26 and causes the fluid to be in a semi-solid or gelatinous state. In the illustrated embodiment, the hydrophilic material 52 comprises hydrophilic beads 54 which are further contained in a second inner bag 56 (embodying herein a second bag located within such first bag; wherein such urine-absorber is contained within such second bag). Preferably, bag 56 comprises a thin external layer of fluid-permeable material which will allow the fluid (urine) to easily pass through it and be absorbed by the hydrophilic beads 54 (this arrangement embodying herein wherein such second bag is fluid permeable). Preferably, use of the hydrophilic material 52 reduces the likelihood of spillage should the disposable urinal 24 be accidentally dropped or the outer bag torn. In addition, bag 56 preferably comprises a deodorant or fragrance which is preferably released upon activation of the hydrophilic material 52 (embodying herein a deodorizer contained within such second bag and structured and arranged to be activated by the urine; and embodying herein a deodorizer contained within such first bag and structured and arranged to be activated by the urine). Such hydrophilic material 52 is readily available by those knowledgeable in the art. Neither the hydrophilic material 52 nor the bag 56 is a required element for the disposable urinal system 20 to function, but both are preferred as a method to reduce the chance of spillage. Those knowledgeable in the art, under appropriate circumstances, may find other suitable methods to reduce spillage without deterring from the present invention.

FIG. 5 is a section view of section 5-5 of FIG. 4 and further illustrates rim 34. Preferably, rim 34 comprises a soft absorbent seal 36 along the outer periphery of the funnel rim 34 that functionally prevents leaking during urine voiding, when held closely to the body of the user 22, between the legs, as shown. Preferably, the soft absorbent seal 36 comprises an inner layer 52 of non-woven fabric, such as cotton, with an outer layer 54 of preferably an exterior cellulose fiber layer 48 (this arrangement embodying herein wherein such rim portion comprises a substantially cotton-fiber material). Under appropriate circumstances, other materials may suffice.

Preferably, the exterior cellulose fiber layer 48 connects along a horizontal seam 56 to the outer layer 44 (which is also liquid and odor impenetrable material, such as plastic) by adhesive means or by mechanical means, such as stitching. Under appropriate circumstances, other connecting methods may suffice. Preferably, the exterior cellulose fiber layer 48 connects along a horizontal seam 58 to the upper inside layer 46 by adhesive means, as shown, or by mechanical means, such as stitching. Under appropriate circumstances, other connecting methods may suffice, such as having the exterior cellulose fiber layer 48 continue as an integral piece downward to the funnel neck portion 30. Also shown in FIG. 5 is the connection between the funnel 28 and bag 26.

FIG. 7 is a section view through section 7-7 of FIG. 2. Preferably, in one embodiment, outer layer 44 is adhesively connected by an adhesive 58 to outer layer 42 of bag 26. In another embodiment, outer layer 44 is connected to outer layer 42 of bag 26 by use of heat-sealing the two preferably plastic pieces together. Preferably, the funnel 28 and bag 26 are fully connected around the bottom perimeter 60 of the funnel neck portion 30, as shown. Under appropriate circumstances, other connecting methods may suffice.

FIG. 7 is a section view through section 7-7 of FIG. 2. FIG. 7 further illustrates the preferred folding of the funnel 28 and the relative location and arrangement of the disposable urinal 24. FIG. 7 further illustrates sliding closure device 40. Preferably, sliding closure device 40 is integrally connected to bag 26 in well-known ways. Preferably, flexible extended portion 31 is also connected along the interior base portion 62 of the sliding closure device 40, as shown.

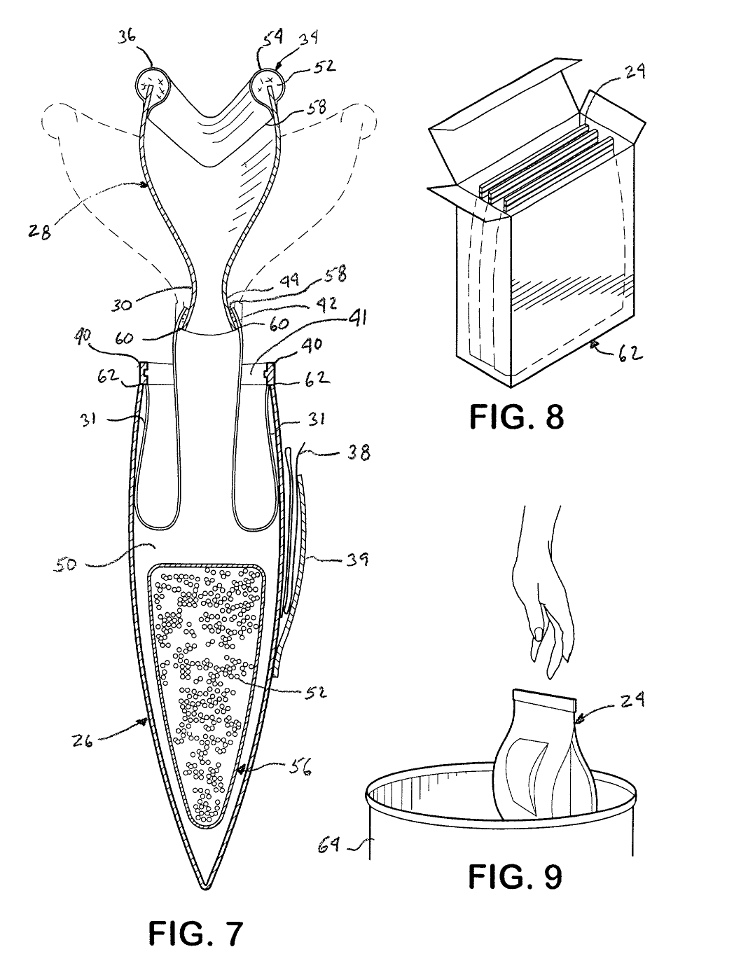

FIG. 8 is a perspective view of the disposable urinal system 20 illustrating three disposable urinals 24 stored in a packaging container 62 (embodying herein kit means for packaging for sale in a single package at least one such holding means, at least one such funnel means, and at least one such removal means; and embodying herein a plurality of products, within one such kit means, each comprising one such holding means, one such funnel means, and one such removal means). Preferably, the disposable urinals 24 are packaged for easy accessability and use.

FIG. 9 is a perspective view illustrating the disposable urinal 24 being disposed in a trash container 64. Under appropriate circumstances, use of materials to manufacture the disposable urinal 24 other than that described, that are recyclable or more easily degradable, may suffice.

In operation, the disposable urinal 24 is packaged for easy accessability and use. Preferably, prior to use, the funnel 28 and rim portion are folded together and stored inside the bag 26, as illustrated in FIG. 6. Preferably, the sliding closure device 40 is in a closed position. For use, the sliding closure device 40 is opened (in well-known ways), and the user 22 reaches into the bag 26 and grasps the funnel 28 and pulls it out of the bag 26 such that the funnel extends above the bag 26, as illustrated in FIG. 7. Preferably, the user 22 then unfolds or opens the funnel 28, extending the rim 34 portion such that it will fit between the user's legs and provide enough of an opening to urinate into. Preferably, the user 22 grasps the funnel neck portion 30 with one hand, such that the hand encompasses the bottom portion of the funnel 28, as illustrated in FIG. 1. Preferably, the user 22 then firmly holds the soft absorbent seal 36 closely to the body of the user 22, between the legs, as shown. Preferably, such that the soft absorbent seal 36 will prevent leaking during urine voiding. After use, the user preferably utilizes the tissue 38 or cleaning wipe, as provided on the exterior of bag 26 in tissue holder 39. Preferably, the tissue 38 is discarded into the funnel 28, and the funnel is folded and pushed into the bag 26 such that the sliding closure device 40 may be positioned in a closed position (the above described arrangement embodying herein wherein such funnel is structured and arranged to wholly fit into such first bag, be pullable through such opening from such first bag into a position to direct flow of the urine from such funnel into such first bag, and be returnable wholly through such opening into such first bag after urination; and embodying herein removal means for implementing extension of such funnel means from such holding means and stowage of such funnel means within such holding means; and also embodying herein a removal system structured and arranged to implement extension of such funnel from such holder for use during urination and stowage of such funnel within such holder before and after). Preferably, the bag 26 is now sealed and ready for disposal at the convenience of the user 22.

Although applicant has described applicant's preferred embodiments of this invention, it will be understood that the broadest scope of this invention includes such modifications as diverse shapes and sizes and materials. Such scope is limited only by the below claims as read in connection with the above specification.

Further, many other advantages of applicant's invention will be apparent to those skilled in the art from the above descriptions and the below claims.

What is claimed is:

1) A disposable, hand-holdable, portable urinal system for holding the urine of a user during and after urination, comprising:
a) a first bag comprising:
i) substantially a liquid- and odor-impenetrable material,
ii) at least one hollow interior, and
iii) at least one opening
(1) comprising a closure which is structured and arranged to seal and un-seal said at least one opening,
iv) wherein said first bag is structured and arranged to hold the urine; and
b) a funnel, attached to an interior of said first bag, comprising
i) a liquid- and odor-impenetrable material, and
ii) an end portion having a rim portion,
iii) wherein said funnel is structured and arranged to
(1) wholly fit into said first bag,
(2) be pullable through said opening from said first bag into a position to direct flow of the urine from said funnel into said first bag, and
(3) be returnable wholly through said opening into said first bag after urination,
iv) wherein said end portion is structured and arranged to permit a single hand of the user to hold said funnel so as to position said rim portion without substantial interference to a flow of the urine through said funnel, and
v) wherein said rim portion
(1) is structured and arranged to closely fit the genitals of a female user and
(2) comprises a soft absorbent seal along a periphery of said rim portion structured and arranged to substantially prevent leaking of the urine, during urination, from said periphery of said rim portion.

2) The system according to claim 1 further comprising a urine-absorber located within said first bag.

3) The system according to claim 2 wherein said urine-absorber is a gellable hydrophilic material.

4) The system according to claim 3 further comprising:
a) a second bag located within said first bag;
b) wherein said urine-absorber is contained within said second bag; and
c) wherein said second bag is fluid permeable.

5) The system according to claim 4 further comprising a deodorizer contained within said second bag and structured and arranged to be activated by the urine.

6) The system according to claim 1 further comprising a deodorizer contained within said first bag and structured and arranged to be activated by the urine.

7) The system according to claim 5 further comprising:
a) a holder attached to an exterior of said first bag,
b) wherein said holder is structured and arranged to hold at least one wiping element.

8) The system according to claim 1 further comprising:
a) a holder attached to an exterior of said first bag,
b) wherein said holder comprises at least one wiping element.

9) The system according to claim 8 wherein said at least one wiping element comprises at least one tissue.

10) The system according to claim 8 wherein said at least one wiping element comprises at least one moistened towelette.

11) The system according to claim 1 wherein said closure comprises a flexible closure.

12) The system according to claim 1 wherein said funnel is foldable.

13) The system according to claim 5 wherein said funnel is foldable.

14) The system according to claim 1 wherein said rim portion comprises a substantially cotton-fiber material.

15) The system according to claim 1 wherein said rim portion comprises a stiffener structured and arranged to substantially maintain shape during urination use.

16) The system according to claim 13 comprising:
a) a holder attached to an exterior of said first bag;
b) wherein said holder comprises at least one wiping element selected from the group consisting of
i) a moist towlette
ii) a wiping tissue; and
c) wherein said rim portion comprises a substantially cotton-fiber material; and
d) wherein said rim portion comprises a stiffener structured and arranged to substantially maintain shape during urination use.

17) A disposable, hand-holdable, portable urinal system for holding the urine of a user, during and after urination, comprising:
a) a sealable holding means for holding the urine;
b) funnel means, attached to an interior of said holding means, for funneling the urine into the holding means;
c) wherein said funnel means comprises rim means for cushioned leak-sealing of said funnel means adjacent genitals of the user during urination; and
d) removal means for implementing
i) extension of said funnel means from said holding means and
ii) stowage of said funnel means within said holding means.

18) The system according to claim 17 further comprising:
a) kit means for packaging for sale in a single package at least one said holding means, at least one said funnel means, and at least one said removal means.

19) The system according to claim 18 comprising a plurality of products, within one said kit means, each comprising one said holding means, one said funnel means, and one said removal means.

20) A disposable, hand-holdable, portable urinal system for holding the urine of a user, during and after urination, comprising:
a) a sealable holder structured and arranged to hold the urine;
b) a funnel, attached to an interior of said holder, structured and arranged to funnel the urine into said holder;
c) wherein said funnel comprises a rim portion structured and arranged to provide cushioned leak-sealing of said funnel adjacent genitals of the user during urination; and
d) a removal system structured and arranged to implement
i) extension of said funnel from said holder for use during urination and
ii) stowage of said funnel within said holder before and after.
